(19) United States
(12) Patent Application Publication
(10) Pub. No.: US 2021/0031056 A1
(43) Pub. Date: Feb. 4, 2021

(54) CHARGED PARTICLE BEAM TREATMENT APPARATUS

(71) Applicant: SUMITOMO HEAVY INDUSTRIES, LTD., Tokyo (JP)

(72) Inventor: Junichi Inoue, Ehime (JP)

(21) Appl. No.: 17/067,036

(22) Filed: Oct. 9, 2020

Related U.S. Application Data
(63) Continuation of application No. PCT/JP2018/015441, filed on Apr. 12, 2018.

(51) Int. Cl. A61N 5/10; G21K 1/04; G21K 5/04

(52) U.S. Cl. CPC A61N 5/1077; A61N 5/1068; A61N 5/1071; A61N 2005/1074; G21K 5/04; A61N 2005/1087; G21K 1/04

(57) ABSTRACT

A charged particle beam treatment apparatus includes: an ion source that generates charged particles; an accelerator that accelerates the charged particles generated in the ion source and emits a charged particle beam; an irradiator that irradiates an irradiation target with the charged particle beam; and a controller that controls the ion source, in which the controller stores operating parameters of the ion source when the irradiation of the irradiation target with the charged particle beam is interrupted, and the controller operates the ion source, based on the stored operating parameters, when the irradiation of the irradiation target with the charged particle beam is resumed.

### RELATED APPLICATIONS

The content of International Patent Application No. PCT/JP2018/015441, on the basis of which priority benefits are claimed in an accompanying application data sheet, is in its entirety incorporated herein by reference.

### BACKGROUND

### Technical Field

Certain embodiments of the present invention relate to a charged particle beam treatment apparatus.

### Description of Related Art

In the past, a charged particle beam treatment apparatus that performs treatment by irradiating an affected part of a patient with a charged particle beam has been known. The related art discloses a charged particle beam generating device that performs scanning with a charged particle beam generated in an ion source and emitted from an accelerator with a scanning electromagnet and then irradiates an affected part of a patient with the charged particle beam.

### SUMMARY

According to an embodiment of the present invention, there is provided a charged particle beam treatment apparatus including: an ion source that generates charged particles; an accelerator that accelerates the charged particles generated in the ion source and emits a charged particle beam; an irradiator that irradiates an irradiation target with the charged particle beam; and a controller that controls the ion source, in which the controller stores operating parameters of the ion source, corresponding to the operating parameters when the irradiation of the irradiation target with the charged particle beam is interrupted, and the controller operates the ion source, based on the stored operating parameters, when the irradiation of the irradiation target with the charged particle beam is resumed.

### BRIEF DESCRIPTION OF THE DRAWINGS

FIG. 1 is a diagram schematically showing a charged particle beam treatment apparatus according to an embodiment of the present invention.

FIG. 2 is a schematic configuration diagram of an irradiator and a controller of the charged particle beam treatment apparatus of FIG. 1.

FIGS. 3A and 3B are diagrams showing layers set with respect to a tumor.

FIGS. 4A to 4C are diagrams for describing an operation of a charged particle beam treatment apparatus according to a comparative example.

FIGS. 5A to 5C are diagrams for describing an operation of the charged particle beam treatment apparatus according to the present embodiment.

### DETAILED DESCRIPTION

Incidentally, there is a case where the position of an affected part which is irradiated with a charged particle beam changes in conjunction with the respiration of a patient. Therefore, there is a case where a method of performing the irradiation with the charged particle beam only at a specific timing of the respiration is used in order to restrain portions other than the affected part from being irradiated with the charged particle beam. However, in such a method, the state of the inside of the accelerator (particularly the ion source) changes while the irradiation of the affected part with the charged particle beam is being stopped, and thus there is a case where the intensity of the charged particle beam which is emitted from the accelerator becomes unstable (the intensity overshoots) when the irradiation is resumed. Therefore, there is a possibility that a desired dose distribution may not be obtained, so that the irradiation with the charged particle beam may not be satisfied with a treatment plan.

It is desirable to provide a charged particle beam treatment apparatus in which it is possible to stabilize the intensity of a charged particle beam.

The controller of the charged particle beam treatment apparatus stores the operating parameters of the ion source when the irradiation of the irradiation target with the charged particle beam is interrupted. Then, the controller operates the ion source, based on the stored operating parameters, when resuming the irradiation of the irradiation target with the charged particle beam. In this way, even in a case where the state of the ion source changes while the irradiation with the charged particle beam is being interrupted, the ion source can be controlled to the same operating parameters as those immediately before the irradiation with the charged particle beam is interrupted. Therefore, it is possible to suppress overshoot of the intensity of the charged particle beam and stabilize the intensity of the charged particle beam.

The charged particle beam treatment apparatus according to an embodiment may further include an intensity measurement unit that measures the intensity of the charged particle beam emitted from the accelerator, and the controller may control the operation of the ion source, based on the intensity of the charged particle beam measured by the intensity measurement unit. According to this configuration, the ion source can be controlled based on the intensity of the emitted charged particle beam, and therefore, it is possible to more effectively stabilize the intensity of the charged particle beam.

In the charged particle beam treatment apparatus according to an embodiment, the irradiator may continuously perform the irradiation with the charged particle beam along a trajectory determined in advance. According to this configuration, even in a case of using a so-called line scanning method which is easily affected by a change in the intensity of the charged particle beam, it is possible to stabilize the intensity of the charged particle beam.

Hereinafter, various embodiments will be described in detail with reference to the drawings. In each drawing, identical or corresponding parts are denoted by the same reference numerals, and overlapping description is omitted.

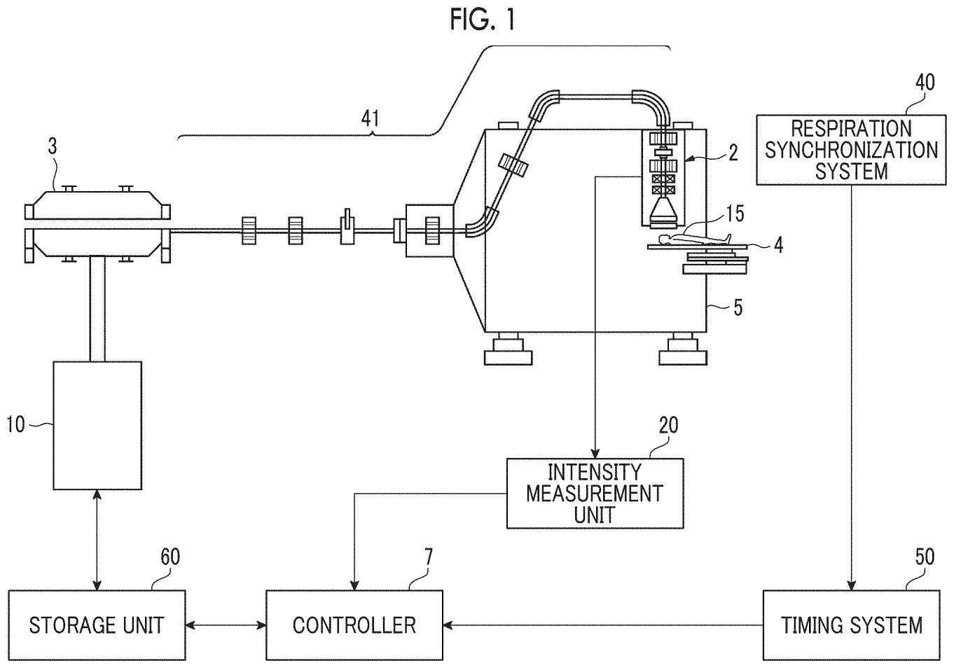

FIG. 1 is a diagram schematically showing the configuration of a charged particle beam treatment apparatus according to an embodiment. A charged particle beam treatment apparatus 1 shown in FIG. 1 is an apparatus that is used for cancer treatment or the like by a radiotherapy method, and includes an ion source 10 that generates charged particles, an accelerator 3 that accelerates the charged particles generated in the ion source 10 and emits a charged particle beam, an irradiator 2 that irradiates a tumor (an irradiation target) of a patient 15 with the charged particle beam, and a controller 7 that controls the entire charged particle beam treatment apparatus 1. Further, the charged particle beam treatment apparatus 1 includes a beam transport line 41 for transporting the charged particle beam emitted from the accelerator 3 to the irradiator 2, an intensity measurement unit 20 that measures the intensity of the charged particle beam emitted from the accelerator 3, a respiration synchronization system 40 that detects the respiration of the patient 15, and a rotating gantry 5 provided so as to surround a treatment table 4. The irradiator 2 is mounted to the rotating gantry 5. The controller 7 has a storage unit 60 that stores the operating parameters of the ion source 10.

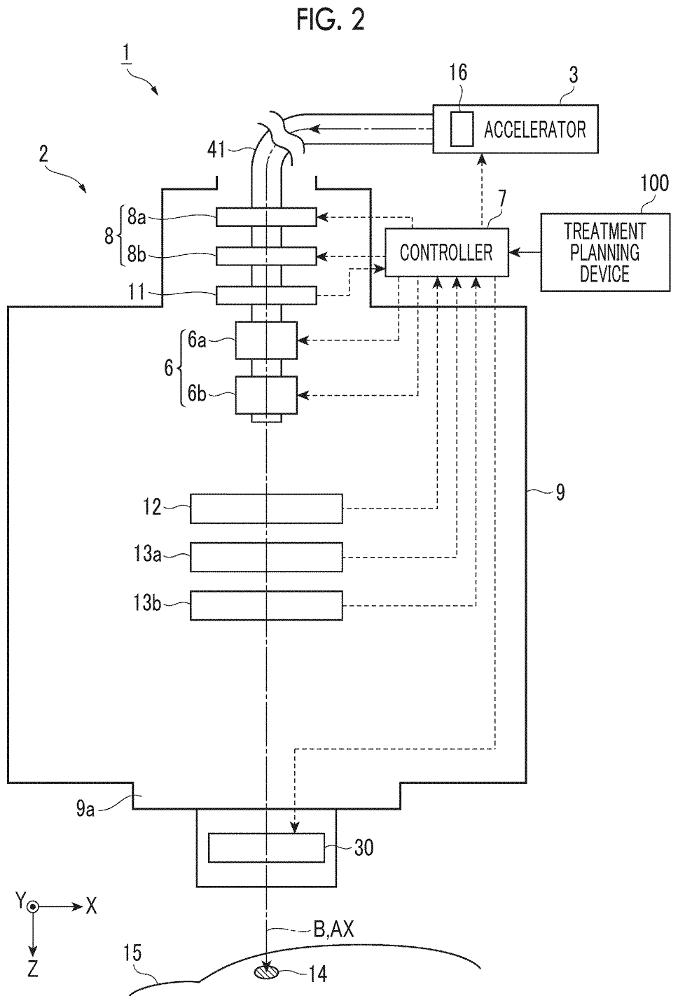

FIG. 2 is a schematic configuration diagram of the irradiator and the controller of the charged particle beam treatment apparatus of FIG. 1. In the following description, the terms “X direction”, “Y direction”, and “Z direction” will be used. The “Z direction” is a direction in which a base axis AX of a charged particle beam B extends, and is an irradiation depth direction of the charged particle beam B. The “base axis AX” is an irradiation axis of the charged particle beam B in a case where it is not changed by a scanning electromagnet 6 (described later). FIG. 2 shows a state where the irradiation with the charged particle beam B is performed along the base axis AX. The “X direction” is one direction in a plane orthogonal to the Z direction. The “Y direction” is a direction orthogonal to the X direction in the plane orthogonal to the Z direction.

First, a schematic configuration of the charged particle beam treatment apparatus 1 according to the present embodiment will be described with reference to FIGS. 1 and 2. The charged particle beam treatment apparatus 1 is an irradiation apparatus related to a scanning method. The scanning method is not particularly limited, and line scanning, raster scanning, spot scanning, or the like may be adopted. As shown in FIG. 2, the charged particle beam treatment apparatus 1 includes the accelerator 3, the irradiator 2, the beam transport line 41, and the controller 7.

The accelerator 3 is a device that accelerates the charged particles generated in the ion source 10 and emits the charged particle beam B. As the accelerator 3, a cyclotron, a synchrotron, a synchrotron, a linac, or the like can be given as an example. The accelerator 3 is connected to the controller 7, and the operation of the accelerator 3 is controlled by the controller 7, whereby the intensity of the charged particle beam B which is emitted is controlled. The charged particle beam B generated in the accelerator 3 is transported to an irradiation nozzle 9 through the beam transport line 41. The beam transport line 41 connects the accelerator 3 and the irradiator 2 and transports the charged particle beam emitted from the accelerator 3 to the irradiator 2. In the present embodiment, the ion source 10 is provided outside the accelerator 3. However, the ion source 10 may be provided inside the accelerator 3.

The charged particle beam treatment apparatus 1 further includes a beam chopper 16 that is disposed in the accelerator 3 and is capable of blocking the charged particle beam B emitted from the ion source 10. The beam chopper 16 blocks the charged particle beam B by deflecting the charged particle beam B to remove it from an acceleration trajectory. In the operating state (ON) of the beam chopper 16, the charged particle beam B emitted from the ion source 10 is blocked, and thus a state is created where the charged particle beam B is not emitted from the accelerator 3. In the stopped state (OFF) of the beam chopper 16, a state is created where the charged particle beam B emitted from the ion source 10 is emitted from the accelerator 3 without being blocked. The operating state and the stopped state of the beam chopper 16 can be switched by a beam chopper switch (not shown). Means other than the beam chopper may be used as means for switching between the irradiation with the charged particle beam and the non-irradiation. For example, a shutter may be provided in the beam transport line 41, and the charged particle beam B may be blocked by the shutter. In this case, the charged particle beam B is blocked by causing the shutter to invade the acceleration trajectory of the charged particle beam B. Alternatively, the charged particle beam B may be emitted from the accelerator 3 only at the time of the irradiation with the charged particle beam B, by using a deflector (electromagnet) provided in the accelerator 3. Further, the charged particle beam B may be blocked by stopping a power supply of the ion source 10.

The irradiator 2 is for irradiating a tumor (an irradiation target) 14 in the body of the patient 15 with the charged particle beam B. The charged particle beam B is obtained by accelerating charged particles at a high speed, and a proton beam, a heavy particle (heavy ion) beam, an electron beam, or the like can be given as an example. Specifically, the irradiator 2 is a device that irradiates the tumor 14 with the charged particle beam B emitted from the accelerator 3 that accelerates the charged particles generated in the ion source 10, and transported through the beam transport line 41. The irradiator 2 includes a scanning electromagnet (scanning unit) 6, a quadrupole electromagnet 8, a profile monitor 11, a dose monitor 12, flatness monitors 13a and 13b, and a degrader 30. The scanning electromagnet 6, the monitors 11, 12, 13a, and 13b, the quadrupole electromagnet 8, and the degrader 30 are accommodated in the irradiation nozzle 9.

The scanning electromagnet 6 includes an X-direction scanning electromagnet 6a and a Y-direction scanning electromagnet 6b. Each of the X-direction scanning electromagnet 6a and the Y-direction scanning electromagnet 6b is composed of a pair of electromagnets, and changes a magnetic field between the pair of electromagnets according to an electric current which is supplied from the controller 7 to perform scanning with the charged particle beam B passing between the electromagnets. The X-direction scanning electromagnet 6a performs the scanning with the charged particle beam B in the X direction, and the Y-direction scanning electromagnet 6b performs the scanning with the charged particle beam B in the Y direction. The scanning electromagnets 6a and 6b are disposed in this order downstream of the charged particle beam B with respect to the accelerator 3 on the base axis AX.

The quadrupole electromagnet 8 includes an X-direction quadrupole electromagnet 8a and a Y-direction quadrupole electromagnet 8b. The X-direction quadrupole electromagnet 8a and the Y-direction quadrupole electromagnet 8b narrow and converge the charged particle beam B according to the electric current which is supplied from the controller 7. The X-direction quadrupole electromagnet 8a converges the charged particle beam B in the X direction, and the Y-direction quadrupole electromagnet 8b converges the charged particle beam B in the Y direction. The beam size of the charged particle beam B can be changed by changing the narrowing amount (convergence amount) by changing the electric current which is supplied to the quadrupole electromagnet 8. The quadrupole electromagnets 8a and 8b are disposed in this order between the accelerator 3 and the scanning electromagnet 6 on the base axis AX. The beam size is the size of the charged particle beam B in an X-Y plane. Further, the beam shape is the shape of the charged particle beam B in the X-Y plane.

The profile monitor 11 detects the beam shape and position of the charged particle beam B for alignment at the time of initial setting. The profile monitor 11 is disposed between the quadrupole electromagnet 8 and the scanning electromagnet 6 on the base axis AX. The dose monitor 12 detects the intensity of the charged particle beam B and transmits a signal to the intensity measurement unit 20. The dose monitor 12 is disposed downstream of the scanning electromagnet 6 on the base axis AX. The flatness monitors 13a and 13b detect and monitor the beam shape and position of the charged particle beam B. The flatness monitors 13a and 13b are disposed downstream of the charged particle beam B with respect to the dose monitor 12 on the base axis AX. Each of the monitors 11, 12, 13a, and 13b outputs the detected detection result to the controller 7.

The degrader 30 performs fine adjustment of the energy of the charged particle beam B by reducing the energy of the charged particle beam B passing therethrough. In the present embodiment, the degrader 30 is provided at a tip portion 9a of the irradiation nozzle 9. The tip portion 9a of the irradiation nozzle 9 is an end portion downstream of the charged particle beam B. The degrader 30 in the irradiation nozzle 9 can be omitted.

The controller 7 includes, for example, a CPU, a ROM, a RAM, and the like. The controller 7 controls the accelerator 3, the scanning electromagnet 6, and the quadrupole electromagnet 8, based on the detection result output from each of the monitors 11, 12, 13a, and 13b. Further, in the present embodiment, the controller 7 feeds back the detection result of each of the monitors 11, 12, 13a, and 13b to control the quadrupole electromagnet 8 such that the beam size of the charged particle beam B becomes constant. Further, the controller 7 controls the operation of the ion source 10 such that the output of the ion source 10 becomes constant, based on the intensity of the charged particle beam B measured by the intensity measurement unit 20.

Further, the controller 7 of the charged particle beam treatment apparatus 1 is connected to a treatment planning device 100 that performs a treatment plan of the charged particle beam treatment apparatus. The treatment planning device 100 measures the tumor 14 of the patient 15 by CT or the like before treatment, and plans a dose distribution (a dose distribution of the charged particle beam for irradiation) at each position of the tumor 14. Specifically, the treatment planning device 100 creates a treatment plan map with respect to the tumor 14. The treatment planning device 100 transmits the created treatment plan map to the controller 7.

In a case of performing the irradiation with the charged particle beam by the scanning method, the tumor 14 is virtually divided into a plurality of layers in the Z direction, and is scanned and irradiated with the charged particle beam in one layer. Then, after the irradiation of the one layer with the charged particle beam is completed, the irradiation of the next adjacent layer with the charged particle beam B is performed.

In a case of performing the irradiation with the charged particle beam B by the scanning method using the charged particle beam treatment apparatus 1 shown in FIG. 2, the quadrupole electromagnet 8 is set to the operating state (ON) so as to converge that the charged particle beam B passing therethrough.

Next, ions are generated in the ion source 10. The ions generated in the ion source 10 are accelerated inside the accelerator 3 and emitted as the charged particle beam B from the accelerator 3. The emitted charged particle beam B performs scanning under the control of the scanning electromagnet 6. In this way, the tumor 14 is irradiated while being scanned with the charged particle beam B within an irradiation range in one layer set in the Z direction. When the irradiation with respect to one layer is completed, the next layer is irradiated with the charged particle beam B.

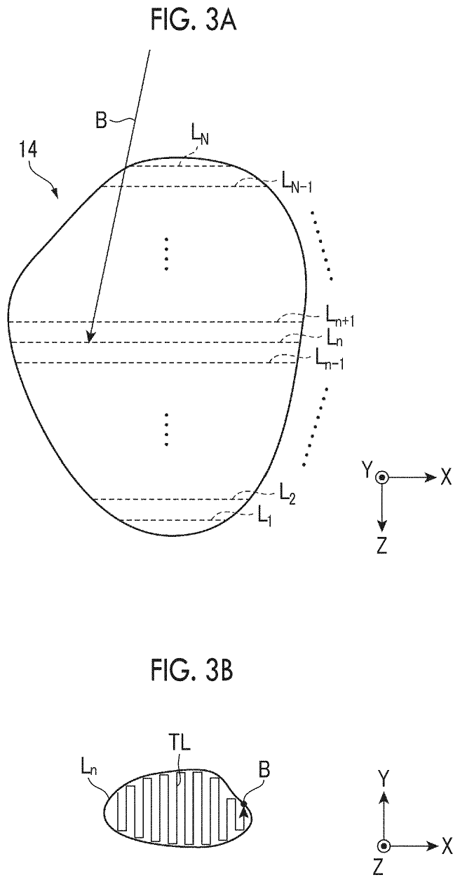

A charged particle beam irradiation image of the scanning electromagnet 6 under the control of the controller 7 will be described with reference to FIGS. 3A and 3B. FIG. 3A shows an irradiation target virtually sliced into a plurality of layers in a depth direction, and FIG. 3B shows a scanning image with the charged particle beam in one layer viewed in the depth direction.

As shown in FIG. 3A, the irradiation target is virtually sliced into a plurality of layers in their radiation depth direction, and in this example, the irradiation target is virtually sliced into N layers such as a layer L₁, a layer L₂, . . . , a layer Lₙ₋₁, a layer Lₙ, a layer Lₙ₊₁, . . . , a layer L_{N-1}, and a layer L_{N} in order from a deep layer (a layer where the range of the charged particle beam B is long). Further, as shown in FIG. 3B, the charged particle beam B performs irradiation of a plurality of irradiation spots of the layer Ln while drawing a beam trajectory TL. That is, the charged particle beam B controlled by the controller 7 moves on the beam trajectory TL.

Next, the respiration synchronization system 40 and the controller 7 will be described in detail with reference to FIG. 1 again. The respiration synchronization system 40 detects the respiration of the patient 15 by using a sensor and generates a gate signal synchronized with the respiration of the patient 15. The gate signal can be generated, for example, by irradiating the abdomen of the patient 15 with a laser light to detect a change in bulge of the abdomen. The gate signal generated in the respiration synchronization system 40 is output to a timing system 50. The timing system 50 determines whether or not to perform the irradiation with the charged particle beam B, based on the gate signal, and generates a pulse signal indicating a timing of the irradiation with the charged particle beam B. The pulse signal generated by the timing system 50 is output to the controller 7. The controller 7 switches between the operating state and the stopped state of the beam chopper 16, based on the pulse signal. In this way, it is possible to switch between an irradiation state where the tumor of the patient 15 is irradiated with the charged particle beam B and an interrupted state where the irradiation of the tumor of the patient 15 with the charged particle beam B is interrupted, according to the respiration of the patient 15. Therefore, it is possible to perform the irradiation with the charged particle beam B only at a specific timing of respiration in order to restrain portions other than the tumor from being irradiated with the charged particle beam B.

The storage unit 60 of the controller 7 stores the operating parameters of the ion source 10 when the irradiation of the tumor of the patient 15 with the charged particle beam B is interrupted. Then, the controller 7 operates the ion source 10 with the operating parameters stored in the storage unit 60, when resuming the irradiation of the tumor of the patient 15 with the charged particle beam B. As the operating parameters of the ion source 10, the electric current and voltage of an arc which is generated in a chimney of the ion source 10, the electric current and voltage flowing through a filament in the chimney, and the like can be given as examples. In the present embodiment, the storage unit 60 is provided outside the controller 7. However, the storage unit 60 may be provided integrally with the controller 7.

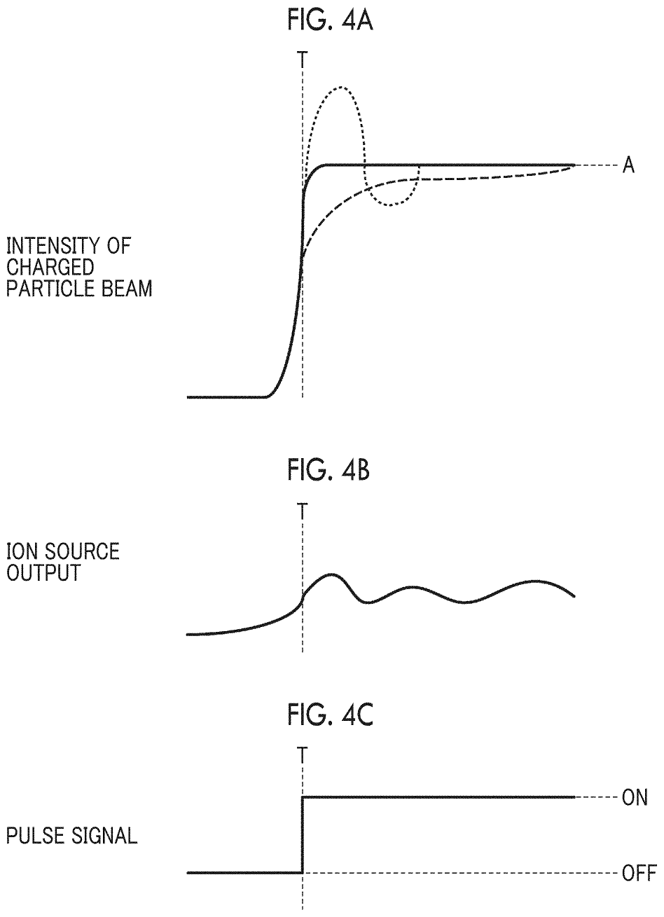

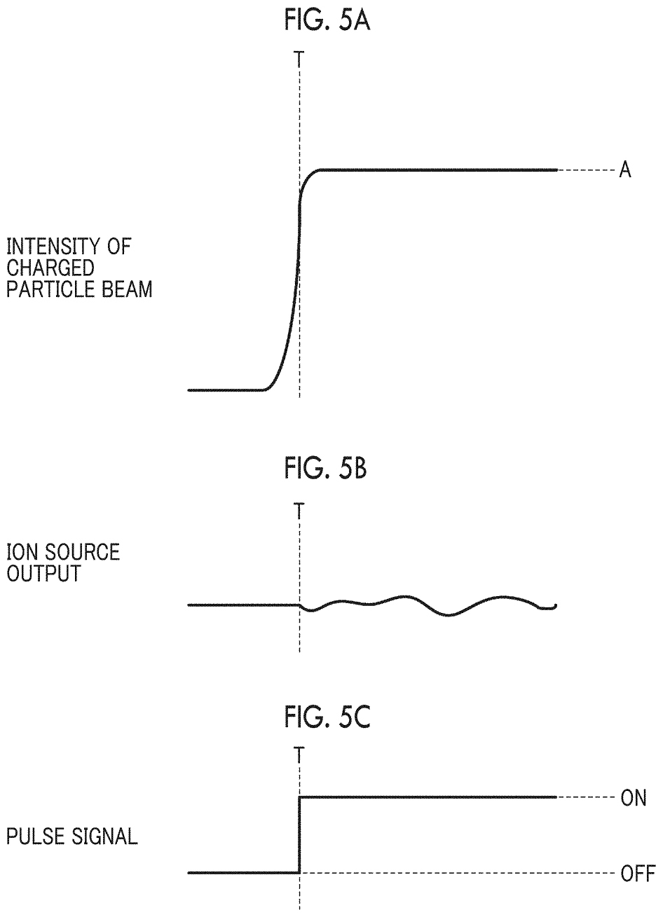

Next, the operation of the charged particle beam treatment apparatus 1 according to the present embodiment will be described with reference to FIGS. 4A to 4C and FIGS. 5A to 5C. FIGS. 4A to 4C are diagrams for describing an operation of a charged particle beam treatment apparatus according to a comparative example. FIGS. 5A to 5C are diagrams for describing an operation of the charged particle beam treatment apparatus according to the present embodiment. FIGS. 4A to 4C and FIGS. 5A to 5C show the intensity of the charged particle beam, the intensity of the output of the ion source, and the timing signal, respectively. In the charged particle beam treatment apparatus according to the comparative example, an initial parameter is set to the ion source, and feedback control is performed based on the intensity of the charged particle beam such that the output of the ion source becomes constant while the tumor of the patient is being irradiated with the charged particle beam. However, since the dose monitor that detects the intensity of the charged particle beam is provided in the irradiator, the charged particle beam cannot be detected by the dose monitor while the irradiation with the charged particle beam is being stopped. Therefore, feedback control is not performed while the irradiation with the charged particle beam is being stopped, and an initial parameter is set to the ion source again at a timing T of resuming the irradiation with the charged particle beam. Therefore, as shown in FIG. 4B, the output of the ion source after the timing T becomes unstable. As a result, as shown in FIG. 4A, there is a case where the intensity of the charged particle beam becomes unstable with respect to a desired intensity A, such as the intensity of the charged particle beam overshooting.

In contrast, in the charged particle beam treatment apparatus 1 according to this example, the storage unit 60 of the controller 7 stores the operating parameters of the ion source 10 when the irradiation with the charged particle beam B is interrupted, and when resuming the irradiation with the charged particle beam B, the controller 7 operates the ion source 10, based on the operating parameters stored in the storage unit 60. Therefore, when the irradiation is resumed, a state close to the state immediately before the operation of the ion source 10 is stopped is created, and therefore, as shown in FIG. 5B, it is possible to stabilize the output of the ion source 10 after the timing T of resuming the irradiation with the charged particle beam B. Further, it is possible to bring the output of the ion source 10 after the timing T close to the output of the ion source 10 before the irradiation with the charged particle beam B is interrupted. Therefore, as shown in FIG. 5A, it is possible to control the intensity of the charged particle beam B to a value close to the desired intensity A and stabilize it.

The controller 7 may perform a predetermined calculation with respect to the operating parameters stored in the storage unit 60 and operate the ion source 10 with the calculated operating parameters when resuming the irradiation with the charged particle beam B. As the predetermined calculation, for example, the controller 7 may add (or subtract) a predetermined value to (from) the operating parameters stored in the storage unit 60, or may multiply the operating parameters by a predetermined coefficient.

As described above, the storage unit 60 of the controller 7 of the charged particle beam treatment apparatus 1 stores the operating parameters of the ion source 10 when the irradiation of the tumor (irradiation target) of the patient 15 with the charged particle beam B is interrupted. Then, the controller 7 operates the ion source 10, based on the operating parameters stored in the storage unit 60, when resuming the irradiation of the tumor (irradiation target) of the patient 15 with the charged particle beam B (at the timing T). In this way, even in a case where the state of the ion source 10 changes while the irradiation with the charged particle beam B is being interrupted, the ion source 10 can be controlled to the same operating parameters as those immediately before the irradiation with the charged particle beam B is interrupted. That is, when the irradiation is resumed, a state close to the state immediately before the operation of the ion source 10 is stopped can be created. Therefore, it is possible to suppress overshoot or the like of the intensity of the charged particle beam B and stabilize the intensity of the charged particle beam B.

Further, the charged particle beam treatment apparatus 1 further includes the intensity measurement unit 20 that measures the intensity of the charged particle beam B emitted from the accelerator 3, and the controller 7 controls the operation of the ion source 10, based on the intensity of the charged particle beam B measured by the intensity measurement unit 20. In this way, the ion source 10 can be controlled based on the intensity of the emitted charged particle beam B, and therefore, it is possible to more effectively stabilize the intensity of the charged particle beam B.

Further, in the charged particle beam treatment apparatus 1, the irradiator 2 continuously performs the irradiation with the charged particle beam B according to the beam trajectory TL determined in advance. In this manner, even in a case of using a so-called line scanning method which is easily affected by a change in the intensity of the charged particle beam B, it is possible to stabilize the intensity of the charged particle beam B.

It should be understood that the invention is not limited to the above-described embodiment, but may be modified into various forms on the basis of the spirit of the invention. Additionally, the modifications are included in the scope of the invention.

What is claimed is:

1. A charged particle beam treatment apparatus comprising:
an ion source that generates charged particles;
an accelerator that accelerates the charged particles generated in the ion source and emits a charged particle beam;
an irradiator that irradiates an irradiation target with the charged particle beam; and
a controller that controls the ion source,
wherein the controller stores operating parameters of the ion source, corresponding to the operating parameters when the irradiation of the irradiation target with the charged particle beam is interrupted, and
the controller operates the ion source, based on the stored operating parameters, when the irradiation of the irradiation target with the charged particle beam is resumed.

2. The charged particle beam treatment apparatus according to claim 1, further comprising:
an intensity measurement unit that measures intensity of the charged particle beam emitted from the accelerator,
wherein the controller controls the operation of the ion source, based on the intensity of the charged particle beam measured by the intensity measurement unit.

3. The charged particle beam treatment apparatus according to claim 1, wherein the irradiator continuously performs the irradiation with the charged particle beam along a trajectory determined in advance.

4. The charged particle beam treatment apparatus according to claim 1, further comprising:
a respiration synchronization system that detects respiration of a patient having the irradiation target and generates a gate signal synchronized with the respiration; and
a timing system to which the gate signal is output.

5. The charged particle beam treatment apparatus according to claim 4, wherein the timing system determines whether or not to perform the irradiation with the charged particle beam, based on the gate signal, and generates a pulse signal indicating a timing of the irradiation with the charged particle beam.

6. The charged particle beam treatment apparatus according to claim 5, wherein the controller resumes the irradiation of the irradiation target with the charged particle beam, based on the pulse signal.

7. The charged particle beam treatment apparatus according to claim 1, wherein the irradiator includes a scanning electromagnet, a quadrupole electromagnet, a profile monitor, a dose monitor, a flatness monitor, and a degrader.

8. The charged particle beam treatment apparatus according to claim 7, wherein the scanning electromagnet includes an X-direction scanning electromagnet and a Y-direction scanning electromagnet, and
the quadrupole electromagnet includes an X-direction quadrupole electromagnet and a Y-direction quadrupole electromagnet.

9. The charged particle beam treatment apparatus according to claim 8, wherein each of the X-direction scanning electromagnet and the Y-direction scanning electromagnet includes a pair of electromagnets, and changes a magnetic field between the pair of the electromagnets according to an electric current which is supplied from the controller to perform scanning with the charged particle beam passing between the electromagnets, and
the X-direction quadrupole electromagnet and the Y-direction quadrupole electromagnet narrow and converge the charged particle beam according to the electric current which is supplied from the controller.

10. The charged particle beam treatment apparatus according to claim 7, wherein the profile monitor detects a beam shape and a position of the charged particle beam for alignment at the time of initial setting,
the dose monitor detects intensity of the charged particle beam,
the flatness monitor detects and monitors the beam shape and the position of the charged particle beam, and
the degrader adjusts energy of the charged particle beam by reducing the energy of the charged particle beam passing through the degrader.

11. The charged particle beam treatment apparatus according to claim 1, wherein the accelerator has a beam chopper capable of blocking the charged particle beam emitted from the ion source.

12. The charged particle beam treatment apparatus according to claim 11, wherein the beam chopper blocks the charged particle beam by deflecting the charged particle beam to remove the charged particle beam from an acceleration trajectory.

13. The charged particle beam treatment apparatus according to claim 1, further comprising:
a beam transport line for transporting the charged particle beam emitted from the accelerator to the irradiator.
